(19) United States
(12) Patent Application Publication
(10) Pub. No.: US 2001/0023364 A1
(43) Pub. Date: Sep. 20, 2001

(54) Cryogenic stimulating device of acupuncture points

(76) Inventor: Moon-Hwi Ahn, Chollabuk-do (KR)

Correspondence Address: FROMMER LAWRENCE & HAUG, 745 FIFTH AVENUE, NEW YORK, NY, 10151, US

(21) Appl. No.: 09/811,997

(22) Filed: Mar. 19, 2001

(30) Foreign Application Priority Data
Mar. 20, 2000 (KR) 2000-13930

(51) Int. Cl. A61F 7/00; A61F 7/12

(52) U.S. Cl. 607/96

(57) ABSTRACT

The present invention relates to a cryogenic stimulating device of acupuncture points. Previous cryogenic stimulating devices of acupuncture points have limitations in maximizing the effect of lowering blood pressure since such devices do not allow the user to control the cooling temperature of the contact rod according to the individual body temperature.

In order to eliminate the drawbacks of the previous cryogenic stimulating devices, the present invention provides a cryogenic stimulating device of acupuncture points with a temperature sensor which senses the cooling temperature of the contact rod in contact with the skin and a temperature controlling means comparing the temperature of the contact rod and the temperature set by the user and then automatically controlling the temperature of the contact rod according to the temperature set by a user to a preferred degree in consideration of his conditions. Consequently, the user can freely set a cooling temperature of the contact rod by using the present inventive cryogenic stimulating device and thus he may obtain the best effect of high blood pressure treatment.

### FIELD OF THE INVENTION

The present invention relates to a stimulating device of acupuncture points provided with a temperature sensor mounted on a contact rod contacting a user's skin and a temperature controlling means which enables the user to freely set the cooling temperature of the contact rod, whereby according to the difference between the temperature set by the user and that sensed by the temperature sensor the temperature of the contact rod may be automatically adjusted. Therefore, by using the present stimulating device, the user may control the temperature of the contact rod to a preferred degree to give the more effective treatment of acupuncture points in consideration of his conditions.

### DESCRIPTION OF THE PRIOR ART

In general, high blood pressure treatments have been largely done with some occidental medicines (e.g. a medicine for internal use, an injection, etc.) and oriental acupuncture, etc. While, the medicines cause side effects and the oriental acupuncture requires expertness, and furthermore, these treatments cannot bring an effect in a short period.

According to the research on the acupuncture points in a human body, the inventor learned that low-temperature stimulus on acupuncture points helps high blood pressure decrease and thus the inventor have completed the present invention of the cryogenic stimulating device of acupuncture points enabling the users to treat conveniently high blood pressure, headache, fever, and the likes.

FIG. 1 is a cross-sectional view of the cooling device of acupuncture points that was disclosed in the previous application by the present inventor. Referring to the previous device, the heat sink body(radiator) (1), which can be handled by ungloved hands, is designed to maximize discharge of heat. For the discharge of heat, multiple cooling fins (13) are regularly distributed in the shape of a cone. It also accompanies the joining groove (14) to connect a cap (5) in the upper end and a trans-body aperture (11) together with an auxiliary aperture (12), via which electric wires may be drawn out.

A cooling element (2) is attached to a center part of the groove (14) formed in the heat sink body (1) wherein the heat radiating surface of the cooling element (2) is to abut on the trans-body aperture (11), and a contact rod (3) provided with a magnet (4) of a globular shape onto the tip of the rod (3) is attached to the cooling element (2).

Next, a cap (5) which is formed in one with an absorber (6) is inserted into the joining groove (14) so that the contact rod (3) is positioned inside the absorber and then the rod may press the acupuncture points.

Also, a suction device (8) connected to the lower end of the heat sink body (1) may compress the absorber (6) onto acupuncture points of a human body by suction through a connection hose (7) and also suck heat radiated from the radiating surface of the cooling element (2).

The electric cords of the cooling element (2) may be drawn out at a side of the bottom part of the heat sink body (1), through the auxiliary aperture (12) and the trans-body aperture (11).

Therefore, the contact rod (3) of the previous cooling device positions on and presses the acupuncture points onto a human body while operating the suction device (8).

At the same time, this not only compresses acupuncture points by the contact rod (3) cooled by the cooling element (2) but also gratifies the stimulation on the acupuncture points by magnetic power generated by the magnet (4) attached to the contact rod (3). Therefore, cold stimulation of the acupuncture points and promotion of blood circulation ware effective at the same time.

However, the effect to lower blood pressure couldn't be maximized because of the uncontrolled electric current using previous cooling devices.

### SUMMARY OF THE INVENTION

In order to overcome the above defects, an object of the present invention is to provide a cryogenic stimulating device of acupuncture points which enables a user to freely control the temperature of the contract rod to give the most effective outcome for treatment on acupuncture points. Particularly, the inventive device is provided with a temperature sensor mounted on the contact rod that senses the cooling temperature of the contact rod to contact with the skin and then by using a temperature controller, the temperature of the contact rod may be automatically adjusted to a predetermined degree based on the comparison between the temperatures set by the user in consideration of his conditions and sensed by the temperature sensor.

Further, the present invention provides an improved cryogenic stimulating device of acupuncture points on which heat sink fins and a fan are mounted under a cooling element in order to increase the cooling effect of the cooling element.

To achieve the objects stated above, the present inventive cryogenic stimulating device of acupuncture points characterized in that the device comprises:

a contact rod which is projected on upper part of a case in order to contact acupuncture points of a human body;

a cooling element which freezes the contact rod according to an input current;

heat sink fins which are placed under the cooling element;

a fan that increases the cooling effect of the cooling element and is placed under the heat sink fins;

a temperature sensor that senses the cooling temperature of the contact rod and is mounted on the contact rod;

a key input unit to set a cooling temperature of the contact rod;

a temperature controller that sends controlling signals to keep the temperature set to the contact rod based on the comparison between the temperatures set by the key input unit and sensed by the sensor; and

an electric current controller that changes electric current provided to the cooling element according to the controlling signals made by the temperature controller.

### BRIEF DESCRIPTION OF THE DRAWINGS

The above and other objects and features of the instant invention will become apparent from the following description of preferred embodiments taken in conjunction with the accompanying drawings, in which:

FIG. 1 is a sectional view of a known stimulating device of acupuncture points,

FIG. 2 is a sectional view of a part of a stimulating device of acupuncture points of the present invention,

FIG. 3 shows the main parts of the device of the present invention,

FIG. 4 is a block diagram showing the controlling means of the inventive device, and

FIG. 5 is a circuit diagram of the controlling means of the present invention.

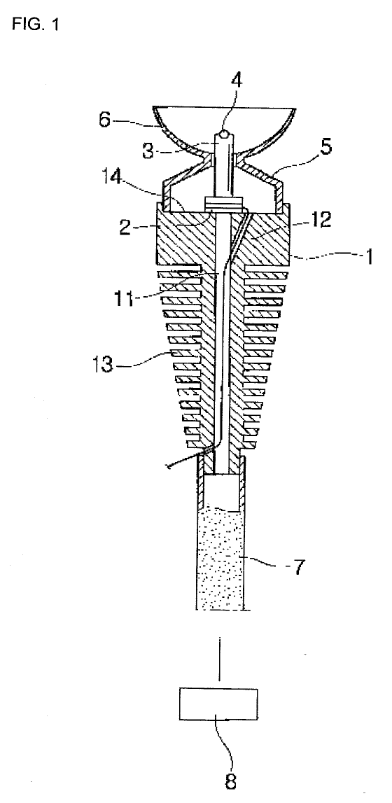

Explanation of symbols of main parts in drawings:

21. Case 22. Contact rod

23. Cooling element 24. Heat sink fins

25. Fan 26. Temperature sensor

27. Key input unit 28. Temperature controller

29. Electric current controller 30. Display

### DETAILED DESCRIPTION OF THE PREFERRED EMBODIMENTS

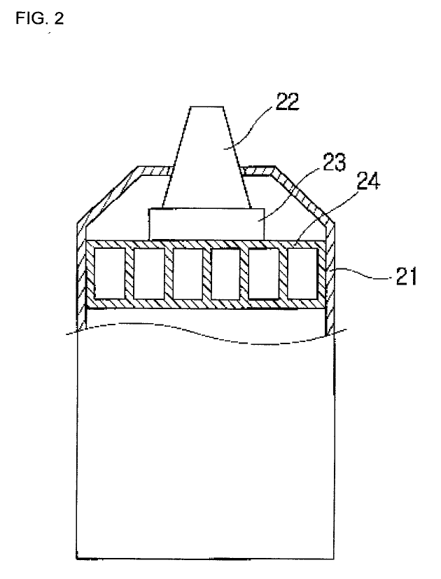

A preferred embodiment of the inventive cryogenic stimulating device of acupuncture points is now described with reference to FIGS. 2 through 5.

A contact rod (22) to contact acupuncture points of a human body is projected upwardly over the upper part of the case (21), while the case forms outward appearance on the whole of the present inventive stimulating device.

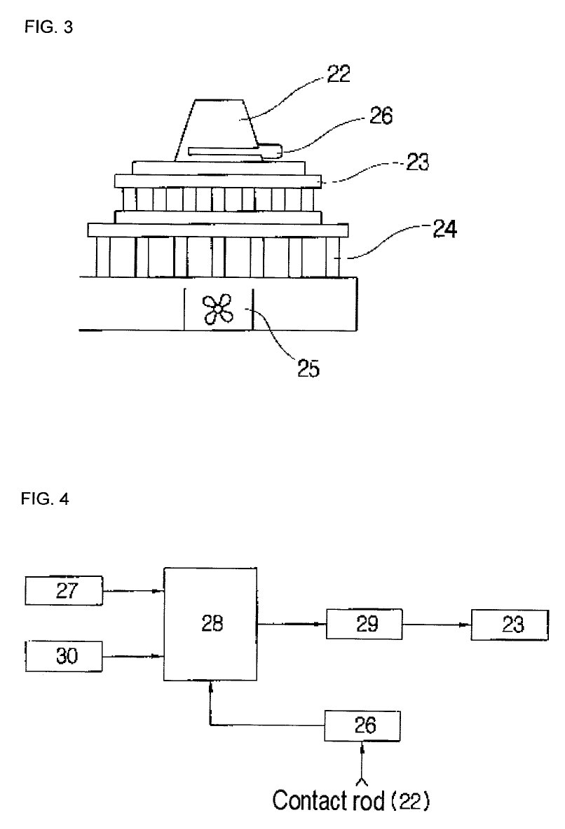

A cooling element (23), of which the upper part faces the contact rod (22) and the lower part faces the heat sink fins (24) under the contact rod (22) as shown in FIG. 3, will freeze the contact rod (22) as it is set according to an input current.

An additional heart sink fins (24) which accompany a fan (25) to increase cooling effect of the cooling element (23) will be assembled under the cooling element (23).

In order words, the cooling element (23) will emit heat absorbed by the cooling board being operated according to an input current; thus, cooling effects will be increased when the heat radiating board should operate smoothly.

Accordingly, the present stimulating device is further provided with a fan (25), whereby the fan (25) blows wind into the heat sink fins (24) which is in contact with the cooling element (23) by the fan (25) to increase the radiation of heat, i.e., the cooling effect.

In addition, the inventive device comprises a controlling means to keep the cooling temperature of the contact rod (22) as the user set. The controlling means comprises the temperature sensor (26) attached to the contact rod to sense the temperature of the contact rod, a key input unit (27) to let the user set the cooling temperature of the contact rod (22), a temperature controller (28) that sends controlling signals in order to maintain the temperature set up by the user according to the result of comparison between the set temperature by the key input unit (27) and the temperature of the contact rod (22) sensed by the temperature sensor (26) and the electric current controller (29) that changes supply of electric current to the cooling element in response to the controlling signals sent by the temperature controller (28). The undefined symbol, 30 in drawings is a display, e.g., a LCD display that indicates the set temperature as the user controls the key input unit (27) and various actions of the device.

Operation or use of the present stimulating device will be explained hereinafter.

First, when should the user enter the desirable cooling temperature by controlling the key input unit (27), the temperature controller (28) emits changed controlling signals to electric current controller (29) to freeze the contact rod (22) at the temperature set . Meantime, the temperature sensor (26) senses the present temperature of the contact rod (22) and sends it to the temperature controller (28). Then, the temperature controller (28) emits changed controlling signals to the electric current controller (29) in response to the difference after comparing temperature set for the contact rod (22) by the user and the present cooling temperature sensed by the temperature sensor (26) while the electric current controller (29) provides the modified current to the cooling element (23) according to the controlling signals changed by the temperature controller (28).

Also, the fan (25) lets heat emitted from heat radiating board of the cooling element (23) and increases the cooling effect of the cooling element (23); thus, the user can set the temperature faster than ever. As it is described above, when the frozen contact rod (22) is placed on the user's acupuncture points, the blood pressure will be lowered; moreover, the user can freely set temperature of the contact rod (22) based on his need.

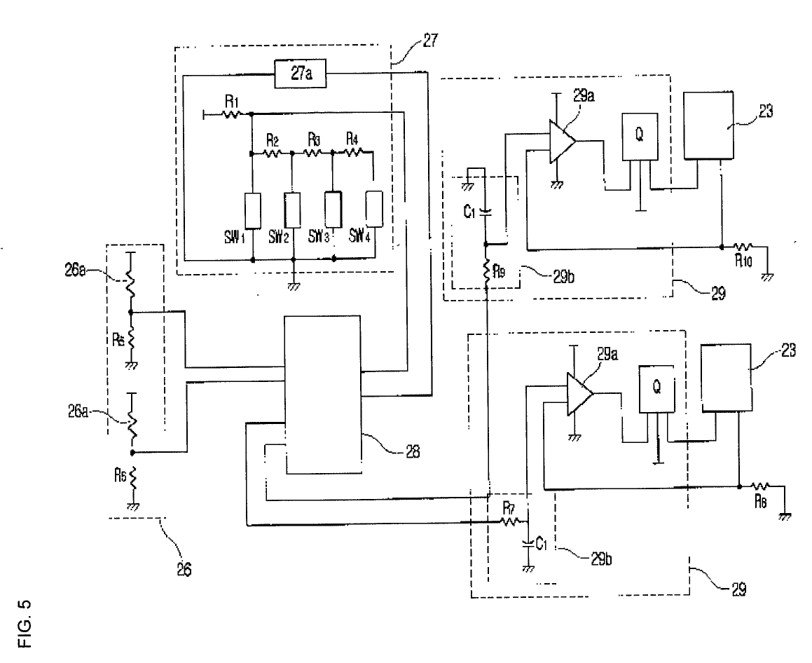

FIG. 5 is a circuit diagram describing a controlling means used in this invention. According to the FIG. 5, the temperature sensor (26) consists of a thermistor (26a) that changes the result as surrounding temperature changes and the resistances (R5,R6). Consequently, when the temperature of the contact rod (22) changes, the result of resistance of the thermistor (26a) will change correspondingly, and then electric current supply sent to the temperature controller (28) will change, whereby the temperature controller (28) may sense the present temperature of the contact rod (22).

The key input unit (27) accompanies multiple switches (SW1˜SW4) and resistances(R1˜R4), and the symbol, 27a is a buzzer.

The electric current controller (29) controls the supply of electric current according to the controlling signals sent from the temperature controller(29). This consists of resistances (R7, R9) and a condensor (C1), a level filter (29b) that changes the controlling signals sent from the temperature controller (29) to read signals, an amplifier (29a) that amplifies signals sent from the level filter (29b), and a transistor (Q) that changes electric current provided to the cooling element (23) according to the signals sent from the amplifier (29a).

In other words, the electrical current controller (29) receives control signals from the low-pass filter (29b), which receives the signals from the temperature controller (28). Once the electrical current controller (29) receives the control signals, it transforms such signals into a certain level of signals in direct current (DC). Then, the signals in direct current may be amplified by the operational amplifier (29a) and supplied to the transistor (Q), then, supplies the current that corresponds to the voltage potential inputted into the transistor to the cooling element (23), thereby effectuating a cooling operation.

More specially, the levels of direct current signals transmitted by the low-pass filter correspond to the signals (pulses) transmitted by the temperature controller (28). The strength of the direct current signals transmitted by the low-pass filter (29b), in turn, controls the cooling temperature of the contact rod (22). The R8 and R10 in the drawings represent electrical resistance.

When using the present inventive stimulating device to give cooling stimulation on acupuncture points, the figures of high blood pressure decreases. For example, after giving treatment of cooling stimulation at temperature ranged between −6. C˜−10. C for 5minutes a day for 2 months to a patient whose blood pressure had averaged 191 dropped to 163. Since the cooling temperature is accordingly adjusted by body temperature, there is no side effect when compressing acupuncture points. Although the present invention is designed to treat high blood pressure, it will bring a great effect to many other problems related to acupuncture points.

### EFFECT OF THE INVENTION

As it is described above repeatedly, the present cryogenic stimulating device of acupuncture points has the sensor to sense the cooling temperature of the contact rod, and moreover, the user can control the temperature of the contact rod, which will bring the greater effect of high blood pressure treatments to lower blood pressure by using low temperature stimulation device on acupuncture points.

1. Cryogenic stimulating device of acupuncture points characterized in that the device comprises:
a contact rod (22) which is projected on the upper part of a case (21) in order to contact acupuncture points of a human body;
a cooling element (23), mounted on the under part of the contact rod (22), which freezes the contact rod according to an input current;
heat sink fins (24) which are placed under the cooling element (23);
a fan (25) that increases the cooling effect of the cooling element and is placed under the heat sink fins (24);
a temperature sensor (26) mounted on the contact rod (22) that senses the cooling temperature of the contact rod (22);
a key input unit (27) to set a cooling temperature of the contact rod (22);
a temperature controller (28) that sends controlling signals to keep the temperature set to the contact rod (22) based on the comparison between the temperatures set by the key input unit (27) and sensed by the sensor (26); and
an electric current controller (29) that changes electric current supplied into the cooling element (23) in response to the controlling signals by the temperature controller (28):

2. The device according to claim 1, characterized in that the temperature controller (28) sends controlling signals in response to the difference between the temperature set to the contact rod by a user and the temperature sensed by the temperature sensor (26), and then the electric current controller (29) changes the electric current in response to the controlling signals by the temperature controller (28).
